(19) United States
(12) Patent Application Publication
(10) Pub. No.: US 2025/0295111 A1
(43) Pub. Date: Sep. 25, 2025

(54) ANTIMICROBIAL PEPTIDES OF ACTINOBACTERIA AND METHODS OF USE THEREOF

(71) Applicant: THE REGENTS OF THE UNIVERSITY OF CALIFORNIA, Oakland, CA (US)

(72) Inventors: Rekha SESHADRI, Berkeley, CA (US); Yasuo YOSHIKUNI, Orinda, CA (US); Natalia IVANOVA, Clayton, CA (US)

(21) Appl. No.: 18/943,590

(22) Filed: Nov. 11, 2024

Related U.S. Application Data
(60) Provisional application No. 63/598,053, filed on Nov. 10, 2023.

(51) Int. Cl. A01N 37/46; A01P 1/00; A01P 3/00; C07K 14/195

(52) U.S. Cl. CPC A01N 37/46; A01P 1/00; A01P 3/00; C07K 14/195

(57) ABSTRACT

The present invention provides for a composition comprising an isolated or purified antimicrobial peptide. The present invention also provides for a method of inhibiting the growth of a fungal or bacterial cell, comprising contacting an antimicrobial peptide, or a composition comprising the isolated or purified antimicrobial peptide, with a fungal or bacterial cell.

### CROSS-REFERENCE TO RELATED APPLICATIONS

This application claims priority to U.S. Provisional Patent Application Ser. No. 63/598,053, filed Nov. 10, 2023, which are hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### REFERENCE TO SEQUENCE LISTING

The instant application contains a Sequence Listing which has been submitted electronically in XML format and is hereby incorporated by reference in its entirety. Said XML copy, created on 15 Apr. 2025, is named “2021-121-02 updated Sequence Listing.xml” and is 131,072 bytes in size.

### FIELD OF THE INVENTION

The present invention is in the field of antimicrobial peptides.

### BACKGROUND OF THE INVENTION

Actinobacteria is a large and diverse phylum comprising Gram-positive bacteria with high guanine-plus-cytosine (G+C) genome content and genome sizes ranging from <0.5 to 15.0 Mbp. Members of this phylum exhibit varying morphological and physiological features, including multicellularity and complex differentiation and are widely (and abundantly) distributed in diverse ecosystems. Famous Actinobacteria include the causative agents of tuberculosis and diphtheria, some of the most devastating diseases in human history. Others play key ecological roles in carbon cycles of soil and aquatic environments or are widespread as mutualistic symbionts of plants and animals, synthesizing natural products for host benefit or helping herbivores digest plant biomass. As renowned producers of diverse secondary metabolites including over two-thirds of all antibiotics in current clinical use and other compounds of clinical or agricultural importance, they are the subject of numerous natural product discovery efforts.

### SUMMARY OF THE INVENTION

The present invention provides for a composition comprising an isolated or purified antimicrobial peptide. In some embodiments, the antimicrobial peptide is obtained or derived from an Actinobacteria.

A family of putative antimicrobial peptides (AMP) was discovered from a subset of Actinobacterial genomes based on whole genome comparisons. The sequences are distantly related to a characterized plant AMP with inhibitory effects against primarily plant fungal phytopathogens. It is hypothesized that this family of bacterial sequences from soil or plant-associated environment will exhibit antimicrobial effects against fungal or gram positive phytopathogens. One of its uses would be in biocontrol of important microbial pathogens of plant crops. In some embodiments, these sequences serve as a novel antimicrobial agent against potential fungal pathogens of plant hosts which may include agriculturally relevant or feedstock crops for biofuels.

In some embodiments, the antimicrobial peptide comprises an amino acid sequence having at least 70%, 80%, 90%, 95%, or 99% amino acid identity with any one of SEQ ID NOs: 1-95. In some embodiments, the antimicrobial peptide has one of the following amino acid sequences:

>2863222795 Ga0436939_03 _588432_588740 [Ga0436939_03:
Nonomuraea muscovyensis DSM 45913]
(SEQ ID NO: 1)
VKRIALVPVISAAMVAIMGSAAYASQYVAYEGPGFTGRSVVIDRCGPSNI
PFHGSYKWYGDGQSGRMYNQRDTQGVMHFELPSNRNAEQRTPVGWQSIFI
VC
>2863339945 Ga0436955_13 _187800_188108 [Ga0436955_13:
Streptosporangium saharense CECT 8840]
(SEQ ID NO: 2)
VKRVLFVSALTAGIVAITTGAAYASQYIAYEGRGFTGRSVVIDACGPSNI
PYHGSYKWYGDGQSGRMYNQPNAQGLMHFKLASDRNAEQRTPVGWQSIFI
EC
>2863381086 Ga0436961_01_1125557_1125865 [Ga0436961_01:
Microbispora chromogenes DSM 43165]
(SEQ ID NO: 3)
MKRFVLVPALTAATIAISAGAAYASQYVAYEGPGFSGRSVVIDRCGVSNI
PFHGSYRWYGDGQSGRMYNQPDAQGVAHFTLAADRNAEQRTPVGWKSIFI
VC
>2516810172 ScinDSM44094_02748 [Sphaerisporangium
cinnabarinum FD25488, DSM 44094]
(SEQ ID NO: 4)
VKRLVLVPVISAAMLTVMAGAAYASQYVAYEGPAFTGRSVVIDRCGPSNI
PFHGSYKWYGDGQSGRMYNQPDTQGVMHFELASNRYAEQRTPVGWKSIFI
VC
>2870519444 Ga0374021_01_2520338_2520646 [Ga0374021_01:
Nonomuraea roseoviolacea DSM 44170]
(SEQ ID NO: 5)
VKRIALVPVIAAAMVAVMGSAAYASQYVAYEGPGFTGRQQFIDACGPSNI
HYHGSYKWYGDGQSGRMYNQPNAQGVMHFELAADRNAEQRTPVGWQSIFI
VC
>2870730807 Ga0374018_01_456203_456511 [Ga0374018_01:
Streptosporangium becharense DSM 46887]
(SEQ ID NO: 6)
MKRIFLVPALTAGIFALTTGAAYASQYIAYEGPGFTGRSVLIDACGPSNI
PYHGSYKWYGDGQSGRMYNQPNAHGVMHFRLPSNNNAEQRTPVGWQSIFI
EC
>2870800808 Ga0373993_01_7200349_7200669 [Ga0373993_01:
Actinomadura catellatispora DSM 44772]
(SEQ ID NO: 7)
MNRKRLALVPALTAGIIACTAAAAQASTIVVYEGSHFSGESMTIDACGPS
DIPERFHGSYKWYATGQSASMYNLPGGKGRVHHRLSAHWDNEQKTPFGWK
SMIIHC
>2891482155 Ga0440672_094_338270_338578 [Ga0440672_094:
Microbispora bryophytorum DSM 46710]
(SEQ ID NO: 8)
MKRFVLVPALTAATIAISAGAAYASQYVAYEGPGFSGRSVVIDRCGMSNI
PFHGSYRWYGDGQSGRMYNQPDAQGVAHFTLAADRNAEQRTPVGWKSIFI
VC
>2676531312 Ga0104469_15129 [Nonomuraea jiangxiensis
CGMCC 4.6533: Ga0104469_151]
(SEQ ID NO: 9)
MKRILLVPAIAAGIIAITAGAAQASQYVAYEGRGFTGRSVVLDACGVSNI
PFHGSYKWYGDGQSGRMYNQPNAQGVAHFTLPSNRNAEQRTPVGWKSIFI
VC
>2676816018 Ga0104451_106371 [Nonomuraea solani CGMCC
4.7037: Ga0104451_106]
(SEQ ID NO: 10)
VKRFVTTAAFTAAIVVGAAGAAYASQYVAYEGPGFTGRSVVIDRCGLSNI
PFNGSYKWYGDGQSGRMYNQPNAEGVAHFTLSSNKNAEQRTPVGWKSIFI
VC
>2682054359 Ga0104720_13735 [Microbispora rosea ATCC
12950: Ga0104720_137]
(SEQ ID NO: 11)
MKRFVLVPALTAATIAISAGAAYASQYVAYEGPGFSGRSVVIDRCGVSNI
PFHGSYRWYGDGQSGRMYNQPDAQGVAHFTLAADRNAEQRTPVGWKSIFI
VC
>2656123332 Ga0075229_1171 [Streptomyces pini PL19:
Ga0075229_117]
(SEQ ID NO: 12)
MRNQHTVRTTVTAAMTAAVLVMSTGAAFASSFRGWSGAHYTGTSSEVTRC
GCSNLSLNHRGSYRFDHTGQDASMENTRNCQGSPHYTFRGDASSPAPVGW
RSIYIHC
>2656124689 Ga0075229_13432 [Streptomyces pini PL19:
Ga0075229_134]
(SEQ ID NO: 13)
MRNQHTVRTTVTAAMTAAVLAVSTGAAFASSFRGWSGAHYTGTSSEVTRC
GCSNLSLGHRGSYRFDHTGQDASMENTRNCQGSPHYTFRGDASSPAPVGW
RSIYIHC
>2574495826 BS75DRAFT_07748 [Streptacidiphilus albus
JL83: BS75DRAFT_unitig_0_quiver.1]
(SEQ ID NO: 14)
MRMFGHRRLLAVGAIAAAVVMGSQAAAFASDFIAYPGPGYSGQAIDLSAC
GYNRIPHGYNGSYVFQYTGQTADAYNNPDASGVAAFRFTGDAQQDTPYGW
QSVWIQC
>2585380296 KF170328 00045 [Streptomyces ansochromogenes:
KF170328_1.1]
(SEQ ID NO: 15)
MKEIVIEMKFTLRTAVALAATAAVSGLAAVPATATEHQARDMTSSFVAYE
GTSFSGRSADINGCGYHSIPFHGSYKWIPRGQDGRMHNAADGRDPVHTVL
RSNRTAEQSTPFGWQSLYIVC
>2629531595 Ga0072930_12546 [Streptomyces sp. CT34:
Ga0072930_1254]
(SEQ ID NO: 16)
MRKFMFSRRAAAVLVATAAIGGAVAAAPATAAESRPAGMTSSFVAYPGTS
FTGQPVDINGCGLHDIPAHGSYKWIARGQSGRMYDQPGAGGPVHTVLASD
ENAEQSTPFGWQSIFIVC
>2635879196 Ga0077416_13276 [Streptomyces sp. NRRL B-
24891: Ga0077416_1327]
(SEQ ID NO: 17)
MRNRHTLRTTVISAVTSAVLALGSGAALASDWYGYPLSNQKGQPQHIRAC
GCSNLAEGKRASYYFDHRGQDATMYNDRDCPRGHGHYKFRGDSERNASFG
WRSIEIHC
>2636589534 Ga0081877_102688 [Streptomyces sp. CMAA 1322:
Ga0081877_102]
(SEQ ID NO: 18)
MRTFTFGRRAAAALVATVAIGGLAAVPSTAAESGAAGMTSSFVAYEGTSF
SGRSADINGCGFHQIPFHGSYKWVPRGQDGRMHNAADGRDPVHTVLRSNR
DAEQSTPFGWQSLYIVC
>2660207415 Ga0115259_104717 [Streptomyces sp. MnatMP-
M17: Ga0115259_10471]
(SEQ ID NO: 19)
MRNRHTLRLAVTSAVTSAALALTSGAALASDWYGYPRSNQQGQPQHILAC
GCSNLDEGKRGSYYFDHRGQDATMENDRDCPRGHGHYTERGDSERNASFG
WKSIHIHC
>2701341501 Ga0139296_117408 [Nonomuraea sp. ATCC 55076:
Ga0139296_11]
(SEQ ID NO: 20)
VKKFVLTSVETAAIVAGTAGAAYASQYVAYEGPGFTGRSVVIDRCGVSNI
PFNGSYKWYGDGQSGRMYNQPNAEGVAHFTLASNRNAEQRTPVGWKSIFI
VC
>2707385817 Ga0123942_10941 [Streptacidiphilus albus
NBRC 100918: Ga0123942_1094]
(SEQ ID NO: 21)
MRMFGHRRLLAVGAIAAAVVMGSQAAAFASDFIAYPGPGYSGQAIDLSAC
GYNRIPHGYNGSYVFQYTGQTADAYNNPDASGVAAFRFTGDAQQDTPYGW
QSVWIQC
>2725993529 Ga0126457_1071132 [Herbidospora
sakaeratensis NBRC 102641: Ga0126457_107]
(SEQ ID NO: 22)
VKRILLTSAIAAVIAGTAGMAHASVYVAYEGPGFSGRSVALSACGINNIP
FGGSYKWYGTGQSGRMYNQPNAQGVAHFTLDSNRNAEQITPVGWKSVFIV
C
>2726013076 Ga0126459_12913 [Herbidospora yilanensis
NBRC 106371: Ga0126459_129]
(SEQ ID NO: 23)
VKRIILTSALAAAVVAGTAGMAHASVYVAYEGPGFSGRSVALSACGTNNI
PFGGSYKWYGTGQSGRMYNQPNAEGTAHFTLDSNRNAEQITPVGWKSIFI
VC
>2726021094 Ga0126460_105739 [Herbidospora daliensis
NBRC 106372: Ga0126460_105]
(SEQ ID NO: 24)
VKRIILTSAIAAVIAGTAGMAHASVYVAYEGPGFSGRSVALSACGINNIP
FGGSYKWYGTGQSGRMYNQPNAEGVAHFTLDSNRNAEQISPVGWKSVFIV
C
>2726025492 Ga0126461_104330 [Herbidospora cretacea NBRC
15474: Ga0126461_104]
(SEQ ID NO: 25)
MKKIILTSALAAAFTLGTAGAALASQYVAYEFPGFAGRSVVISACGMSNI
PFHGSYKWYGDGQSGRMYNQPNAQGVAHFTLDSARNAEQRTPVGWQSIFI
VC
>2768393350 Ga0162434_10285 [Streptomyces scopuliridis
RB72 NRRL B-24574: Ga0162434_1028]
(SEQ ID NO: 26)
MRNRRNRRTLRSTVTATAVAGVLALGAGTAVASGELSWEGPQNTGRAKEL
TRCGCSNIDPNFRAAYRFTYTGQTAAMYNQPDCAGTSHYTFRGNSESVQP
FGWRSIFIHC
>2772814839 Ga0162436_102984 [Streptosporangium
amethystogenes NRRL B-2639: Ga0162436_1029]
(SEQ ID NO: 27)
VKRIALVPVISAAMVAVMGSAAYASQYVAYEGPGFTGRSVVIDRCGPSNI
PFHGSYKWYGDGQSGRMYNQPNTQGVTHFELPSNRNAEQRTPVGWKSIFI
VC
>2772919577 Ga0162428_104249 [Herbidospora cretacea NRRI
B-16917: Ga0162428_104]
(SEQ ID NO: 28)
VKRILLTSALAAVIAGTAGMAHASVYVAYEGPGFSGRSVALSACGINNIP
FGGSYKWYGTGQSGRMYNLPNAQGVAHFTLDSNRNAEQITPVGWKSVFIV
C
>2777257409 Ga0163368_102075 [Streptomyces lilacinus
NRRL B-1968: Ga0163368_10207]
(SEQ ID NO: 29)
MLNSKFSRRAAAALAATAVIGGLTAVPATATESRAYGMTSSFVAYEGTSY
SGRSADINGCGYHSIPFHGSYKWIPRGQDGRMHNAADGRDPVHTVLRSNR
DAEQSTPFGWQSIYIVC
>2792320219 Ga0309290_1010101 [Nonomuraea indica DRQ-2:
Ga0309290_1010]
(SEQ ID NO: 30)
VKRLALVPVISAAMVAIMGTAAYASQYVAYEGPGFTGRSVVIDRCGPSNI
PFHGSYKWYGDGQSGRMYNQRDTQGVMHFELPSDRNAEQRTPVGWQSIFI
VC
>2794041654 Ga0308705_11032 [Streptomyces lushanensis
NRRL B-24994: Ga0308705_1103]
(SEQ ID NO: 31)
MRNRHTLRLAVTSAVTSAVLALTSGAALASDWYGYPQSNQKGQPQHILAC
GCSNLDEGKRGSYYFDHRGQDATMENDRDCPRGHGHYTFRGDSERNASFG
WKSIHIHC
>2794152429 Ga0309198_105512 [Streptomyces barkulensis
RC 1831: Ga0309198_1055]
(SEQ ID NO: 32)
MRNQHTVRTTVIAAMTAAVLAVSTGAAFASSFRGWSGAHYTGTSSEVTGC
GCSNLSIGHRGSYRFDHTGQDASMENTRNCQGSPHYTFRGDASSPAPVGW
RSIYIHC
>2839802719 Ga0336955_824 [Ga0336955_013: Microbispora
sp. GKU 823]
(SEQ ID NO: 33)
MKRFVLVPALTAATIAISAGAAYASQYVAYEGPGFSGRSVVIDRCGVSNI
PFHGSYRWYGDGQSGRMYNQPDAQGVAHFTLAADRNAEQRTPVGWQSIFI
VC
>2840024097 Ga0396303_10429 [Ga0396303_81: Nonomuraea
sp. WAC 01424]
(SEQ ID NO: 34)
MKKVLLVPAIAAGIVLASAGAAYASQYVAYEGPGFTGRSVVLDACGVSNI
PYHGSYKWYGDGQSGRMYNQPNATGVTHFELASNRNAEQRTPVGWKSIFI
VC
>2840999734 Ga0337170_10439 [Ga0337170_27: Actinomadura
parvosata kistnae ERS1554153]
(SEQ ID NO: 35)
VKKFVLTSVETAAIVAGTAGAAYASQYVAYEGPGFTGRSVVIDRCGVSNI
PFNGSYKWYGDGQSGRMYNQPNAEGVAHFTLASNRNAEQRTPVGWKSIFI
VC
>2866652978 Ga0437514_176_3572_3895 [Ga0437514_176:
Actinomadura sp. KC216]
(SEQ ID NO: 36)
MKARHAVRSAVTASVAAAVVVLGTGSAYASSFLSWEGPQNTGRAKVVHAC
GCNNIDDNFKAAYRFTYTGQTAAMYNEPDCKGLVHHTFRGSQESKDPFGW
RSIFFHC
>2866684946 Ga0437518_083_10152_10472 [Ga0437518_083:
Actinomadura sp. 7K534]
(SEQ ID NO: 37)
MNRKRLALLPALTAGIIACTAAAAQASTIVVYEGSHFTGASMTIDACGPS
DIPERFHGSYKWYATGQSASMYNLPGGKGRVHHKLSAHWDNEQKTPFGWK
SMIIHC
>2866821255 Ga0437535_217_1204_1524 [Ga0437535_217:
Actinomadura sp. H3C3]
(SEQ ID NO: 38)
VRKKTMALVPALTAGIIAVSAAAAQASTIVVYEGDHYSGRSMTINKCGMS
NIPEAFHGSYKWYATGQSARMYNQPNAEGVMHYELSARHNNEQATPFGWK
SMFIVC
>2866884700 Ga0437543_354_46831_47136 [Ga0437543_354:
Nonomuraea sp. 6K102]
(SEQ ID NO: 39)
MSLVLVPALAAGLITFAPTTAQASTFVVYEGSHYTGRSAALSACGMSNIP
YNGSYKWYATGQSARMYNQPNGVGVGHYTFSASRSGERPEGFGWKSIIIH
C
>2866904020 Ga0437546_11_202890_203213 [Ga0437546_11:
Streptomyces sp. WAC05374]
(SEQ ID NO: 40)
MRIRRTLRTAVTSAVMTGVFVLGTGVASASSFLSWEGPQNTGRAKEVFAC
GCNNIDPNFRAAYRFKHTGQDATMYNQDNCQGQSHFTFHGSQESRDPFGW
RSVFFHC
>2867720352 Ga0436988_052_8163_8486 [Ga0436988_052:
Streptomyces aurantiacus NRRL ISP-5412]
(SEQ ID NO: 41)
MRTRHALRTAVSATMLAGALLLGTSPAFASSFLSWAGPQNTGSAKEVFAC
GCNNIDPGHKAAYRFTYTGQTAAMYNESNCQGTVHHTFRGSQESRDGFGW
KSVFFHC
>2872793660 Ga0438854_01_7875938_7876261 [Ga0438854_01:
Streptomyces niveus SCSIO 3406]
(SEQ ID NO: 42)
MRKRHALRTAVSATMLAGALVLGSGTAIASSFLSWSGPQNTGSAREVFAC
GCNEIDPGHKAAYRFTYTGQTAAMYNEEGCRGKVHTTFRGSQESRGAFGW
KSVFFHC
>2890732023 Ga0440565_076_18348_18653 [Ga0440565_076:
Herbidospora galbida NEAU-GS14]
(SEQ ID NO: 43)
VKRIILTSALAAVIAGTAGIAHASVYVAYEGPGFSGRSVALTACGTSNIP
FGGSYKWYGTGQSGRMYNQPNAEGTAHFTLDSNRNAEQVTPVGWKSIFIV
C
>2890742602 Ga0440913_02_267702_268007 [Ga0440913_02:
Herbidospora sp. NEAU-GS84]
(SEQ ID NO: 44)
VKRILLTSALAAVIAGTAGMAHASVYVAYEGPGFSGRSVALSACGINNIP
FGGSYKWYGTGQSGRMYNQPNAQGVAHFTLDSNRNAEQITPVGWKSVFIV
C
>2891398803 Ga0442715_11_632923_633231 [Ga0442715_11:
Microbispora catharanthi CR1-09]
(SEQ ID NO: 45)
MRRFVLVPALTAATIAISAGAAYASQYVAYEGPGFSGRSVVIDRCGVSNI
PFHGSYRWYGDGQSGRMYNQPDAQGVAHFTLAADRNAEQRTPVGWKSIFI
VC
>2891540017 Ga0442743_140_257765_258073 [Ga0442743_140:
Microbispora sp. KK1-11]
(SEQ ID NO: 46)
MRRIVLIPALTAATIAISAGAAYASQYVAYEGPGFSGRSVVIDRCGVSNI
PFHGSYRWYGDGQSGRMYNQPDAQGVAHFTLAADRNAEQRTPIGWKSIFI
VC
>2894490164 Ga0444183_45_258790_259098 [Ga0444183_45:
Microbispora sp. Gxj-6]
(SEQ ID NO: 47)
MRRIALVPALTAATIVATAGAAYASQYVAYEGPGFSGRSVVIDACGTSNI
PYHGSYRWYGDGQSGRMYNQPNAQGVAHFTLPSDRNAEQRTPVGWQSIFI
VC
>2895463912 Ga0441209_068_120977_121285 [Ga0441209_068:
Nonomuraea sp. FMUSA5-5]
(SEQ ID NO: 48)
VKKFVLTSVETAAIVAGTAGAAYASQYVAYEGPGFTGRSVVIDRCGVSNI
PFNGSYKWYGDGQSGRMYNQPNAQGVAHFTLASNRNAEQRTPVGWKSIFI
VC
>2895482426 Ga0442701_188_6285_6623 [Ga0442701_188:
Nonomuraea sp. 160415]
(SEQ ID NO: 49)
VKSIKRIALVSAISASLVTIMGGAANASISAAPGSMYVAYEGPGFTGRTQ
VISQCGTTNLRYRGSYKWYGSGQAGRMHNQINARGPVHFKLPTKGNAQQN
KAVGWKSIFKVC
>2895485444 Ga0442701 336_8978_9286 [Ga0442701_336:
Nonomuraea sp. 160415]
(SEQ ID NO: 50)
MKRVTLIPALTAGIIALTAGAAHASQYIAYEGPGFTGRSVLIDACGMSNI
PFNGSYKWYGDGQSGRMYNQPNAEGLMHFKLPADNSAEQRTPVGWKSIFI
EC
>2908281228 Ga0439583_01_681269_681592 [Ga0439583_01:
Streptomyces ficellus NRRL 8067]
(SEQ ID NO: 51)
MRIRRTLRTAITSTVMTGVFVLGTGVASASSFLSWEGPQNTGRAKEVFAC
GCNNIDPNFRAAYRFKHTGQDATMYNQDNCQGQSHFTFHGSQESRDPFGW
RSVFFHC
>2911048110 Ga0477457_1079_757_1065 [Ga0477457_1079:
Microbispora sp. CLES2]
(SEQ ID NO: 52)
MKRYVLVPALTAATIAISAGAAYASQYVAYEGPGFSGRSVVIDRCGVSNI
PFHGSYRWYGDGQSGRMYNQPDAQGVAHFTLAADRNAEQRTPVGWKSIFI
VC
>2911065372 Ga0477332_20_61702_62010 [Ga0477332_20:
Microbispora sp. H10830]
(SEQ ID NO: 53)
MKRFVLVPALTAATIAISAGAAYASQYVAYEGPGFSGRSVVIDRCGVSNI
PFHGSYRWYGDGQSGRMYNQPDAQGVAHFTLAADRNAEQRTPVGWKSIFI
VC
>2920545494 Ga0477430_11_688135_688443 [Ga0477430_11:
Nonomuraea sp. SMC257]
(SEQ ID NO: 54)
VKRIALVPVLAAAMVAVMGSAAYASQYIAYEGPGFTGRQQFIDACGPSNI
HYHGSYKWYGDGQSGRMYNQPNAQGVMHFELASNRNAEQRTPVGWQSIFI
VC
>2920567620 Ga0477431_22_88865_89173 [Ga0477431_22:
Nonomuraea rhodomycinica TBRC6557]
(SEQ ID NO: 55)
VKRIALVPVLAAAMVAVMGSAAYASQYIAYEGPGFTGRQQFIDACGPSNI
HYHGSYKWYGDGQSGRMYNQPNAQGVMHFELAADRNAEQRTPVGWQSIFI
VC
>2920585361 Ga0477912_07_3221_3877 hypothetical protein
[Ga0477912_07: Nonomuraea sp. SYSU D8015]
(SEQ ID NO: 56)
VLRWQARAVGARGDRQDREEWLEIDRSQRKHAHRGSRLEAFYASESTVLR
VLQAAGMTVVAPIVSVVPTGNCPQRDQSDTLATQLRPMARKLLGRLHAVA
LLSPCVSIEWGLGRIIVKKFILTSAFTAAIVVGTAGAAYASQYVAYEGPG
FTGRSVVIDRCGVSNIPINGSYKWYGDGQSGRMYNQPNAEGVAHFTLASN
RNAEQRTPVGWKSIFIVC
>2929691128 Ga0498203_14_142137_142463 hypothetical
protein [Ga0498203_14: Actinobacteria sp. 002018]
(SEQ ID NO: 57)
MRNRHTLRLAVTSAVTSAVLALTSGAALASDWYGYPLSNQKGQPQHIVAC
GCSNLNEGKRGSYYFDHRGQDATMYNDRDCPRGHGHYTFRGDSERNASFG
WKSIEIHC
>2930772174 Ga0499658_20_104219_104527 hypothetical
protein [Ga0499658_20: Microbispora sp. 049019]
(SEQ ID NO: 58)
MKRFFLVPALTAATIAISAGAAYASQYVAYEGPGFSGRSVVIDRCGMSNI
PFHGSYRWYGDGQSGRMYNQPDAQGVAHFTLAADRNAEQRTPVGWKSIFI
VC
>2931182166 Ga0499646_01_422314_422622 hypothetical
protein [Ga0499646_01: Actinomadura sp. 049627]
(SEQ ID NO: 59)
LKKILLVPAITAGFVLASAGAAYASQYVAYEGPGFTGRSVVLDACGVSNI
PYHGSYKWYGDGQSGRMYNQPNASGVMHFELASNRNAEQRTPVGWKSIFI
VC
>2931650806 Ga0498199_2_185632_185940 hypothetical
protein [Ga0498199_20]
(SEQ ID NO: 60)
LKKILLVPAITAGFVLASAGAAYASQYVAYEGPGFTGRSVVLDACGVSNI
PYHGSYKWYGDGQSGRMYNQPNASGVMHFELASNRNAEQRTPVGWKSIFI
VC
>2931678853 Ga0498222_21_31617_31931_hypothetical
protein [Ga0498222_21]
(SEQ ID NO: 61)
MRKRSLILVPALAAGLITCSATTAQASTFVVYEGSHYTGRSVGLNACGMS
NIPYNGSYKWYATGQSARMYNQPNGQGVGHYTFSGSRSGERPEGFGWKSI
IIHC
>2931971169 Ga0499708_23_133481_133789 predicted
amidohydrolase [Ga0499708_23]
(SEQ ID NO: 62)
VKRIALVPVISAALVAVMGSAAYASQYVAYEGPGFTGRSAIIDRCGPSNI
PFHGSYKWYGDGQSGRMYNQPDAQGVMHFELASDRNAEQRTPVGWKSIFI
VC
>2932289593 Ga0499714_016_136287_136595 hypothetical
protein [Ga0499714_016]
(SEQ ID NO: 63)
VKRIALVPLISAAMVVVMGGAAYASQYVAYEGPGFTGRSVVIDRCGPSNI
PFHGSYKWYGDGQSGRMYNQPDAQGVTHFELASNRNAEQRTPVGWKSIFI
VC
>2933046106 Ga0498332_21_108153_108464 hypothetical
protein [Ga0498332_21]
(SEQ ID NO: 64)
MLKKILLVPAITAGLVLASAGAAYASQYVAYEGRGFTGRSVVLDACGVSN
IPYHGSYKWYGDGQSGRMYNQPNATGVMHFELASNRNAEQRTPVGWKSIF
IVC
>2933136177 Ga0498369_07_293182_293490 hypothetical
protein [Ga0498369_07]
(SEQ ID NO: 65)
MKKVLLVPAIAAGIVLASAGAAYASQYVAYEGPGFTGRSVVLDACGVSNI
PYHGSYKWYGDGQSGRMYNQPNATGVTHFELASNRNAEQRTPVGWKSIFI
VC
>2933147964 Ga0498370_17_56317_56625 hypothetical
protein [Ga0498370_17]
(SEQ ID NO: 66)
VKRIALVPVISAALVVVMGSAAYASQYVAYEGPGFTGRSVVIDRCGPSNI
PFHGSYRWYGDGQSGRMYNQPDTQGVMHFELASNRNAEQRTPVGWKSIFI
VC
>2933235391 Ga0498399_22_57152_57460 hypothetical
protein [Ga0498399_22]
(SEQ ID NO: 67)
MKRIAQVITAAAMTVVATAGTAVASQYVAYEGEGFSGRSVVITACGTSNI
PYHGSYKWYGDGQSGRMYNQSNAQGVAHFTLASNRYAEQRTPVGWKSIFI
VC
>2933338044 Ga0498418_28_88038_88346 hypothetical
protein [Ga0498418_28]
(SEQ ID NO: 68)
VKRIALVPVLAAAMVAIMGSAAYASQYIAYEGPGFTGRTQFIDACGVSNI
VYHGSYKWFGDGQSGRMYNQPNARGPMHFELASNRNAEQHTSVGWQSIFI
VC
>2933458914 Ga0499639_21-61502_61810 hypothetical
protein [Ga0499639_21]
(SEQ ID NO: 69)
VKRIALVPVISAALVVVMGSAAYASQYVAYEGPGFTGRSVVIDRCGPSNI
PFHGSYKWYGDGQSGRMYNQPDTQGVMHFELASNRNAEQRTPVGWKSIFI
VC
>2933489429 Ga0499647_056_29787_30095 hypothetical
protein [Ga0499647_056]
(SEQ ID NO: 70)
MKRILLVPALTAGIIALTAGAAYASQYIAYEGPGFTGRSVLIDACGPSNI
PFHGSYKWFGDGQAGRMYNQPNTQGPMHFRLPADDGAEQRTPVGWQSIFI
EC
>2934292289 Ga0498634_21_175340_175648 hypothetical
protein [Ga0498634_21]
(SEQ ID NO: 71)
LKKILLVPAITAGFVLASAGAAYASQYVAYEGPGFTGRSVVLDACGVSNI
PYHGSYKWYGDGQSGRMYNQPNASGVMHFELASNRNAEQRTPVGWKSIFI
VC
>2934394950 Ga0498263_05_206762_207100 hypothetical
protein [Ga0498263_05]
(SEQ ID NO: 72)
VKSIKKIALVSAISASLITIMGGAANASTSAAPDSMYVAYEGPGFTGRTQ
VITECGLTNLRYRGSYKWYGAGQSGRMHNQINARGSVHFTLPTNSNAQQK
TGIGWKSIFKVC
>2934397538 Ga0498263_09_152335_152643 hypothetical
protein [Ga0498263_09]
(SEQ ID NO: 73)
VKRILLVPALTAGIIALTASAAYASQYVAYEGRGFTGRSVVLDACGVSNI
PFHGSYKWYGDGQSGRMYNQPNAQGVMHFELPSNRNAEQRTPVGWQSIFI
VC
>2934940885 Ga0498643_04_323442_323750 predicted
amidohydrolase [Ga0498643_04]
(SEQ ID NO: 74)
VKKFVLTSVETAAIVAGTAGAAYASQYVAYEGPGFTGRSVVIDRCGVSNI
PFNGSYKWYGDGQSGRMYNQPNAEGVAHFTLASNRNAEQRTPVGWKSIFI
VC
>2934954361 Ga0498646_10_27652_27960 predicted
amidohydrolase [Ga0498646_10]
(SEQ ID NO: 75)
MKRFVLVPALTAATIAISAGAAYASQYVAYEGPGFSGRSVVIDRCGVSNI
PFHGSYRWYGDGQSGRMYNQPDAQGVAHFTLAADRNAEQRTPVGWKSIFI
VC
>2935056647 Ga0498462_05_293238_293546 hypothetical
protein [Ga0498462_05]
(SEQ ID NO: 76)
VKRTLIVSALTAGIVAITAGAAYASQYVAYEGPGFTGRSVVIDACGPSNI
PYHGSYKWYGDGQSGRMYNQPDAQGLMHFRLPSDDNAEQRTPVGWQSIFI
EC
>2936108055 Ga0498599_03_414041_414349 hypothetical
protein [Ga0498599_03]
(SEQ ID NO: 77)
LKKILLVPAITAGFVLASAGAAYASQYVAYEGPGFTGRSVVLDACGVSNI
PYHGSYKWYGDGQSGRMYNQPNASGVMHFELASNRNAEQRTPVGWKSIFI
VC
>2941450566 Ga0498311_07_256920_257312 hypothetical
protein [Ga0498311_07]
(SEQ ID NO: 78)
MEHSLRRRPRSRTRLRATCLALFGAAAIAAPVAIASPANAAPAPARLVLS
ATSTFVAYSGGSFSGDSQGIQGCGSHTIRYHGSYKWYANGQSGHLENNSN
TSGVPHTRLASDDDAESRSGFAWRSILIVC
>2941455497 Ga0498311_28_39630_39959 hypothetical
protein [Ga0498311_28]
(SEQ ID NO: 79)
VKRIILVSAVATALVTAMSGAAYASTSAGYSSTYVAFEGPGFTGRTQVIT
RCGTTNIRYRGSYKWFGGGQDGRMHNDINARGPVHFQLASNRNARQETGI
GWKSIFKVC
>2963020343 Ga0532222_62_5882_6184 hypothetical protein
[Ga0532222_62]
(SEQ ID NO: 80)
VRPVVAAGATAALFVVGTGMAFASRFESYEKANYSGAASTVAACGCNSIE
YGGSYIFEYSGQTARVYKQKGCKGRAAGDLTHNSRNPDTVGGWKSIFILC
>2989286602 Ga0487049_006_16869_17177 hypothetical
protein [Ga0487049_006]
(SEQ ID NO: 81)
VKRLALVPVISAAMIALMGTAAYASQYVAYEGPAFTGRSVVIDRCGPSDI
PFHGSYKWYGDGQSGRMYNQPGTHGVMHFELASNRYAEQRTPVGWQSIFI
VC
>2989465800 Ga0489578_223_112378_112767 hypothetical
protein [Ga0489578_223]
(SEQ ID NO: 82)
MENSHRRRPRSGTRLRATCLALLGAASIATPLIASPANAVSAPARLALSA
TSTFVAYSGSSFSGDSEGIQGCGLHTISYHGSYKWYANGQSGHLENNSTG
SGVPHTRLASDDNAESGSGFAWGSILIVC
>2989479499 Ga0487045_22_133562_133870 predicted
amidohydrolase [Ga0487045_22]
(SEQ ID NO: 83)
VKRFLLVPAMTAAIIAITAGAAYASQYIAYEGRGFTGRSVVIDACGVSNI
PENGSYKWYGDGQSGRMYNQRDAKGVMHFELASNRNAEQRTPVGWKSIFI
VC
>2995213228 Ga0558830_036_145016_145324 hypothetical
protein [Ga0558830_036]
(SEQ ID NO: 84)
MKRVAFVSAATAALVAIMGSAAFASQYVAYEGPGFSGRSVTIDKCGTSNI
PFHGSYKWYGDGQSGRMYNQPNAEGVMHFELASNRYAEQRTPVGWQSIFI
VC
>2997503691 Ga0449348_01_91826_92149 hypothetical
protein [Ga0449348_01]
(SEQ ID NO: 85)
MRHQHTVRTTVTAAVTAAVLVMSTGAAFASSFRGWSGAHYTGTSSEVTRC
GCSNLSLNHRGSYRFDHTGQDASMENTRNCQGSPHYTFRGDSSSPAPVGW
RSIYIHC
>3002160184 Ga0520911_009_131969_132277 hypothetical
protein [Ga0520911_009]
(SEQ ID NO: 86)
MKRFLLVSALTAATIAISAGAAYASQYVAYEGPAFTGRSVVIDKCGVSNI
PFHGSYRWYGDGQSGRMYNQPNAEGVAHFTLPSDRNAEQRTPVGWKSIFI
VC
>3006566871 Ga0493593_019_46964_47317 hypothetical
protein [Ga0493593_019]
(SEQ ID NO: 87)
MPKFAFGRRAAAALVATVAIGGLSAVPSTAAEAGADGMTSSFVAYEGTSF
SGRSADINGCGYHQIPFHGSYKWVPRGQDGRMHNAADGRDPVHTVLRSNR
GAEQSTPFGWQSIYIVC
>8008656206 Ga0594564_048_67936_68259 hypothetical
protein [Ga0594564_048]
(SEQ ID NO: 88)
MRKRRSLRTAVGSLMLSGALVLGTGTSFASSFLSWEGPQNTGRAKEVHAC
GCNNIDPNYRAAYRFTYTGQTAAMYNEPDCRGLVHHTFRGSQESKDAFGW
RSVFFHC
>8008763730 Ga0594709_180_40970_41278 opacity protein-
like surface antigen [Ga0594709_180]
(SEQ ID NO: 89)
MKRFPVALAAAAMSVVAAAGSAAASQLVVYEGEGYSGRSHVITACGASNI
PYHGSYKWYGDGQSGRMYNQPNAQGVAHFTLPSDRNAGQRTPIGWRSIFI
VC
>8045842237 Ga0615671_18_111092_111412 hypothetical
protein [Ga0615671_18]
(SEQ ID NO: 90)
MRKKTLALAPAVTAGIIALSAAAAQASTIVVYEGDHFSGHSITLSKCGMS
NIPARYHGSYKWYATGQSARMYNQPNAEGVVHFVLRSDRNAEQATPFGWR
SMFIEC
>8048052782 Ga0615455_067_53519_53827 hypothetical
protein [Ga0615455_067]
(SEQ ID NO: 91)
MKRILLVPALTAGIIALTASAAYASQYIAYEGRGFTGRSVVLDACGVSNI
PFHGSYKWYGDGQSGRMYNQPNAQGVMHFELPSNRNAEQRTPVGWQSIFI
VC
>8048899153 Ga0615933_039_46740_47048 opacity
protein-like surface antigen [Ga0615933_039]
(SEQ ID NO: 92)
MKRVSLVLAAAAMSVVAAAGSAAASQLVVYEGEGESGRSAVISACGTSNL
PYHGSYKWYSDGQSGRMYNQRDAQGVANFTLAPDRNAEQRTSVGWQSIFI
VC
>8053526129 Ga0615832_20_306624_306932 hypothetical
protein [Ga0615832_20]
(SEQ ID NO: 93)
MKKLAVVPVISAAMLAVMASAAYASQYVAYEGQAFTGRSVVIDRCGVSNI
PFGGSYKWYGDGQSGRMYNQPNAEGVMHFELRSNRNAEQRTPVGWKSIFI
VC
>8057031925 Ga0616974_072_50633_51004 hypothetical
protein [Ga0616974_072]
(SEQ ID NO: 94)
MIIPRTAQQATSIRRVAAALAAAAAIGGLTAVPATATESRAYGMTSSFVA
YEGTSFSGRSADINGCGQHAIPFHGSYKWIPRGQDGRMHNAADGRDPVHT
VLRSNRDAEMSTPFGWQSIYIVC
>2866542668 Ga0437487_384_681_1004 hypothetical
protein [Ga0437487_384: Streptomyces sp. WAC05374]
(SEQ ID NO: 95)
MRIRRTLRTAVTSAVMTGVFVLGTGVASASSFLSWEGPQNTGRAKEVFAC
GCNNIDPNFRAAYRFKHTGQDATMYNQDNCQGQSHFTFHGSQESRDPFGW
RSVFFHC

In some embodiments, the antimicrobial peptide comprises one or more, or all, of the following amino acid sequences: AAYASQY (SEQ ID NO:96), AAYASQYXAYEGPXFTGR (SEQ ID NO:97), HGSYKWYGDGQSGRMYNQ (SEQ ID NO: 98), IDXCGXSNIXXHGSYKWYGDGQSGRMYNQ (SEQ ID NO:99), AEQRTPVGW (SEQ ID NO:100), and/or AEQRTPVGWXSIFIXC (SEQ ID NO:101), wherein X is any amino acid residue.

In some embodiments, the antimicrobial peptide comprises one or more, or all, of the following amino acid sequences: AAXAS (SEQ ID NO:102), SYXF, and/or GQXA, wherein X is any amino acid residue.

In some embodiments, the antimicrobial peptide comprises one or more, or all, of the following amino acid sequences: AXXAAV (SEQ ID NO: 103), AAXASXF (SEQ ID NO: 104), GSYXF (SEQ ID NO:105), TGQXA (SEQ ID NO:106), and/or GDAXXXXPXGWXSXXI (SEQ ID NO:107), wherein X is any amino acid residue.

In some embodiments, the antimicrobial peptide is about 100 amino acid residues to about 220 amino acid residues long. In some embodiments, the antimicrobial peptide is about 100 amino acid residues to about 130 amino acid residues long. In some embodiments, the antimicrobial peptide is about 100 amino acid residues to about 120 amino acid residues long.

In some embodiments, the antimicrobial peptide is any antimicrobial peptide described herein. In some embodiments, the antimicrobial peptide is capable of killing or inhibiting growth of a fungal or bacterial species. In some embodiments, the bacterial species is a Gram-positive bacterial species. In some embodiments, the antimicrobial peptide is capable of killing or inhibiting growth of a broad array of plant pathogenic fungal or bacterial species.

In some embodiments, the antimicrobial peptide has an amino acid sequence comprising SEQ ID NO:96, 98, or 100, and having at least 70% amino acid identity with one of SEQ ID NO:1-6, 8-11, 20, 25, 27, 30, 33-35, 45-48, 50, 52-56, 58-60, 62-71, 73-77, 81. 83, 84, 86, 89, 91, or 93.

In some embodiments, the antimicrobial peptide has an amino acid sequence comprising SEQ ID NO:96, and having at least 70% amino acid identity with one of SEQ ID NO: 1-6, 8, 10, 11, 20, 27, 30, 33-35, 45-48, 52-56, 58-60, 62-66, 68, 70, 71, 73-77, 81. 83, 86, 91, or 93.

In some embodiments, the antimicrobial peptide has an amino acid sequence comprising SEQ ID NO:98, and having at least 70% amino acid identity with one of SEQ ID NO: 1, 2, 4-6, 9, 25, 27, 30, 34, 54, 55, 59, 60, 62-65, 67, 69, 71, 73, 76, 77, 81, 84, 89, or 91.

In some embodiments, the antimicrobial peptide has an amino acid sequence comprising SEQ ID NO:100, and having at least 70% amino acid identity with one of SEQ ID NO: 1-6, 8-11, 20, 25, 27, 30, 33-35, 45, 47, 48, 50, 53-56, 58-60, 62-67, 69-71, 73-77, 81, 83, 84, 86, 91, or 93.

The present invention provides for a pharmaceutical composition comprising the antimicrobial peptide, and a pharmaceutically acceptable carrier.

The present invention provides for a medicant manufactured using the composition of the present invention.

The present invention provides for a nucleic acid encoding the antimicrobial peptide operatively linked to a promoter. In some embodiments, the promoter is heterologous to the antimicrobial peptide.

The present invention provides for a method of inhibiting the growth of a fungal or bacterial cell, comprising contacting the antimicrobial peptide of the present invention, or a composition comprising the isolated or purified antimicrobial peptide, with a fungal or bacterial cell.

The present invention provides for a modified host cell comprises one or more genes encoding, and/or capable of expressing, the antimicrobial peptide.

The present invention provides for a modified host cell, such as a bacterial cell, comprising one or more genes encoding, and/or capable of expressing, the antimicrobial peptide, wherein the naturally occurring unmodified host cell and pathogenic to a subject but is modified to be not pathogenic to the organism.

In some embodiments, the subject is a plant or a mammal, such as a human. In some embodiments, the subject is known to be, suspected to be, or has a high probability of being infected or contaminated with a pathogenic fungus or bacteria. In some embodiments, the subject is a human patient.

The present invention provides for a method of treating a subject suffering, or is suspected of suffering, from a disease caused all or in part by a fungal or bacterial cell, comprising: administering a composition comprising the antimicrobial peptide to a subject.

The present invention provides for a method of treating a disease caused all or in part by a fungal or bacterial cell, comprising: administering a pharmaceutical composition or medicant of the present invention to a subject in need thereof. In some embodiments, the bacterial cell is a human pathogen and the subject is a human patient.

In some embodiments, the bacterial cell is a species from a genus selected from the group consisting of Escherichia, Enterococcus, Staphylococcus, Klebsiella, Acinetobacter, Pseudomonas, and Enterobacter.

In some embodiments, the bacterial cell is an Escherichia coli, Enterococcus faecium, Enterobacter cloacae, Enterobacter aerogenes, Staphylococcus aureus, Klebsiella pneumonia, Acinetobacter baumannii, or Pseudomonas aeruginosa.

The present invention provides for a method to limit or reduce growth of a pathogenic fungi or bacteria in an environment, comprising: introducing a non-pathogenic bacterial comprising one or more genes encoding, and/or capable of expressing, the antimicrobial peptide to an environment; whereby expression of the antimicrobial peptide limits or reduces growth of a pathogenic fungi or bacteria in the environment.

In some embodiments, the environment is an intensive care unit (ICU), or is known to be, suspected to be, or has a high probability of being infected or contaminated with a pathogenic fungus or bacteria.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Functional adaptations of host versus environmental Actinobacteria. Maximum likelihood tree of eukaryal and bacterial candidate sequences assigned to PF09117. Characterized plant reference sequences are highlighted with green text. Bacterial branches are colored red, plant branches are green, and fungal branches are black.

FIG. 2. Inhibition of Saccharomyces cerevisiae by AMP candidate of Streptosporangium becharense DSM 46887 overexpressed in E. coli.

FIG. 3. SDS-PAGE gel showing the overexpression of recombinant AMP in E. coli. Lanes are protein size marker (M), control strain (C), and AMP-producing strain (AMP), respectively. The expected 11.2 kDa band of the AMP is highlighted.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

A polynucleotide or amino acid sequence is “heterologous” to an organism or a second polynucleotide or amino acid sequence, or a promoter, if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety, or a gene that is not naturally expressed in the target tissue).

The term “antimicrobial peptide” comprises salts of the antimicrobial peptide. Salts of the antimicrobial peptide are also provided. Such salts include, but are not limited to, acid addition salts and base addition salts. As used herein, “pharmaceutically acceptable salt” of the antimicrobial peptide refers to a salt that retains the desired antimicrobial, antibacterial, antifungal, antiviral, antiparasitic and/or anti-inflammatory activity of the antimicrobial peptide, and is suitable for administration to humans or animals. Methods for the preparation of salts of the antimicrobial peptide are known in the art and generally involve mixing of the antimicrobial peptide with a pharmaceutically acceptable acid or base, for instance, by reacting the free acid or free base forms of the product with one or more equivalents of the appropriate acid or base in a solvent or medium in which the salt is insoluble, or in a solvent such as water, which is then removed in vacuo or by freeze-drying, or by exchanging the cations of an existing salt for another cation on a suitable ion exchange resin. Examples of pharmaceutically acceptable acids and bases include organic and inorganic acids such as formic acid, acetic acid, propionic acid, lactic acid, glycolic acid, oxalic acid, pyruvic acid, succinic acid, maleic acid, malonic acid, trifluoroacetic acid, cinnamic acid, sulfuric acid, hydrochloric acid, hydrobromic acid, nitric acid, perchloric acid, phosphoric acid, and thiocyanic acid, which form ammonium salts with free amino groups of the antimicrobial peptide, and bases that form carboxylate salts with free carboxylic groups of the antimicrobial peptide, such as ethylamine, methylamine, dimethylamine, triethylamine, isopropylamine, diisopropylamine, and other mono-, di- and trialkylamines, and arylamines.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

SEQ ID NO:6 is an experimentally validated sequence from Streptosporangium becharense DSM 46887. SEQ ID NO:6 is a 102 amino acid peptide with a signal peptide. In some embodiments, the AMP is an at least about 100 amino acid sequence with a signal peptide. The peptide stats for this particular sequence are available in the webpage of img.jgi.doe.gov/cgi-bin/m/main.cgi?section-GeneDetail&page=pepstats&gene_oid=2870730807 (hereby incorporated by reference).

Further information about the AMP is found in: (1) Seshadri et al., “Expanding the genomic encyclopedia of Actinobacteria with 824 isolate reference genomes,” Cell Genomics 2, 100213, 2022 (hereby incorporated by reference), and (2) the webpage for ebi.ac.uk/interpro/entry/pfam/PF09117/(hereby incorporated by reference).

In some embodiments, the antimicrobial peptide exhibits a number of activities that can be advantageously used in both therapeutic and nontherapeutic applications. In some embodiments, the antimicrobial peptide is useful in counteracting various microbial infections, such as bacterial infections, fungal infections, viral infections, and in counteracting parasitic infections. Provided, thus, are pharmaceutical compositions comprising the antimicrobial peptide or a pharmaceutically acceptable salt thereof, and at least one pharmaceutically acceptable carrier, diluent and/or excipient. Also provided are pharmaceutical compositions comprising a nucleic acid molecule or vector of the present invention and at least one pharmaceutically acceptable carrier, diluent and/or excipient.

The present invention also provides for the use of the antimicrobial peptide as a medicament. Further provided is a nucleic acid molecule comprising a nucleic acid sequence encoding the antimicrobial peptide for use as a medicament. The medicament can be a therapeutic or a prophylactic agent.

The present invention also provides for a method for the treatment of a subject suffering from bacterial, fungal, viral and/or parasitic infection comprising administering to the subject a therapeutically effective amount of the antimicrobial peptide, a pharmaceutical composition of the present invention, or a nucleic acid molecule of the present invention. Also provided is a method for the preparation of a medicament for the treatment of a subject infected with a microbe or for prophylaxis of a microbial infection. In some embodiments, the microbe is a bacterium, a fungus, a virus or a parasite. Further provided is the antimicrobial peptide and/or nucleic acid molecule for use in the treatment of a microbial, bacterial, fungal, viral and/or parasitic infection or a condition resulting from a microbial, bacterial, fungal, viral and/or parasitic infection.

As used herein, a “subject” is a human or an animal. Subjects include, but are not limited to, mammals such as humans, pigs, ferrets, seals, rabbits, cats, dogs, cows and horses, and birds such as chickens, ducks, geese and turkeys. In some embodiments, the subject is a mammal. In some embodiments, the subject is a human.

In some embodiments, the pharmaceutical composition of the present invention comprises at least one pharmaceutically acceptable carrier, diluent or excipient. Examples of suitable carriers, for instance, comprise keyhole limpet haemocyanin (KLH), serum albumin (e.g. BSA or RSA) and ovalbumin. In some embodiments, the suitable carrier is a solution, for example, saline. Examples of excipients that can be incorporated in tablets, capsules and the like are the following: a binder such as gum tragacanth, acacia, corn starch or gelatin; an excipient such as microcrystalline cellulose; a disintegrating agent such as corn starch, pregelatinized starch, alginic acid and the like; a lubricant such as magnesium stearate; a sweetening agent such as sucrose, lactose or saccharin; and a flavoring agent such as peppermint, oil of wintergreen or cherry. When the dosage unit form is a capsule, it may contain, in addition to materials of the above type, a liquid carrier such as fatty oil. Various other materials may be present as coatings or to otherwise modify the physical form of the dosage unit. For instance, tablets may be coated with shellac, sugar or both. A syrup or elixir may contain the active compound, sucrose as a sweetening agent, methyl and propyl parabens as preservatives, a dye and a flavoring such as cherry or orange flavor. In some embodiments, the pharmaceutical composition is suitable for human use.

The pharmaceutical compositions described herein can be administered in a variety of different ways. Examples include administering a pharmaceutical composition comprising the antimicrobial peptide and containing a pharmaceutically acceptable carrier via oral, intranasal, rectal, topical, intraperitoneal, intravenous, intramuscular, subcutaneous, subdermal, transdermal, intrathecal, and intracranial methods. For oral administration, the active ingredient can be administered in solid dosage forms, such as capsules, tablets, and powders, or in liquid dosage forms, such as elixirs, syrups, and suspensions.

Sterile compositions for injection can be formulated according to conventional pharmaceutical practice by dissolving or suspending the antimicrobial peptide in a vehicle for injection, such as water or a naturally occurring vegetable oil like sesame oil, coconut oil, peanut oil, cottonseed oil, or the like, or a synthetic fatty vehicle like ethyl oleate or the like. Buffers, preservatives, antioxidants and the like may also be incorporated.

In some embodiments, the pharmaceutical composition is formulated for topical administration. “Topical administration” as used herein refers to application to a body surface such as the skin or mucous membranes to locally treat conditions resulting from microbial or parasitic infections. Examples of formulation suitable for topical administration include, but are not limited to, a cream, gel, ointment, lotion, foam, suspension, spray, aerosol, or powder aerosol. Topical medicaments can be epicutaneous, meaning that they are applied directly to the skin. Topical medicaments can also be inhalational, for instance, for application to the mucosal epithelium of the respiratory tract, or applied to the surface of tissues other than the skin, such as eye drops applied to the conjunctiva, or ear drops placed in the ear. The pharmaceutical composition formulated for topical administration may comprises at least one pharmaceutical excipient suitable for topical application, such as an emulgent, a diluent, a humectant, a preservative, a pH adjuster and/or water.

### REFERENCES CITED HEREIN

1. Verly, R. M., Resende, J. M., Junior, E. F. C., de Magalh˜aes, M. T. Q., Guimar˜aes, C. F. C. R., Munhoz, V. H. O., Bemquerer, M. P., Almeida, F. C. L., Santoro, M. M., Pilo′-Veloso, D., and Bechinger, B. (2017). Structure and membrane interactions of the homodimeric antibiotic peptide homotarsinin. Sci. Rep. 7, 40854. 3
2. Sathoff, A. E., and Samac, D. A. (2019). Antibacterial activity of plant defensins. Mol. Plant Microbe Interact. 32, 507-514.
3. Brogden, K. A. (2005). Antimicrobial peptides: pore formers or metabolic inhibitors in bacteria? Nat. Rev. Microbiol. 3, 238-250.
4. Rima, M., Rima, M., Fajloun, Z., Sabatier, J. M., Bechinger, B., and Naas, T. (2021). Antimicrobial peptides: a potent alternative to antibiotics. Antibiotics 10, 1095. \
5. Zhang, L. J., and Gallo, R. L. (2016). Antimicrobial peptides. Curr. Biol. 26, R14-R19.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Antimicrobial Peptide Cloning and Inhibition Assay

The phylum Actinobacteria includes important human pathogens like Mycobacterium tuberculosis and Corynebacterium diphtheriae and renowned producers of secondary metabolites of commercial interest, yet only a small part of its diversity is represented by sequenced genomes. 824 actinobacterial isolate genomes are presented in the context of a phylum-wide analysis of 6,700 genomes including public isolates and metagenome-assembled genomes (MAGs). It is estimated that only 30%-50% of projected actinobacterial phylogenetic diversity possesses genomic representation via isolates and MAGs. A comparison of gene functions reveals novel determinants of host-microbe interaction as well as environment-specific adaptations such as potential antimicrobial peptides. Plasmids and prophages are identified across isolates and uncover extensive prophage diversity structured mainly by host taxonomy. Analysis of >80,000 biosynthetic gene clusters reveals that horizontal gene transfer and gene loss shape secondary metabolite repertoire across taxa. It is observed that the essential role of and need for high-quality isolate genome sequences.

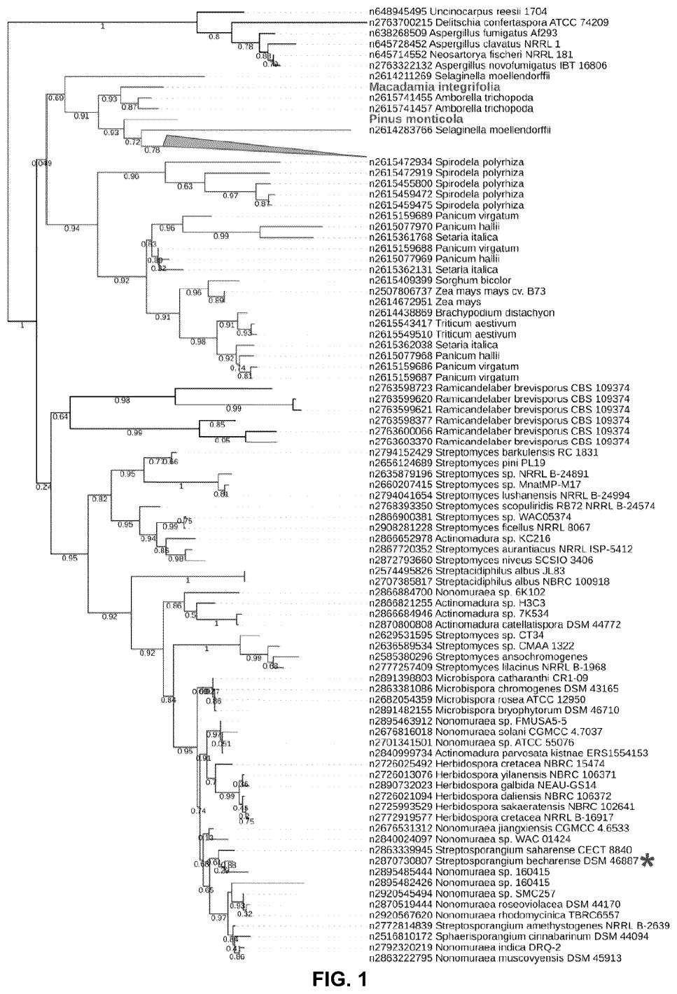

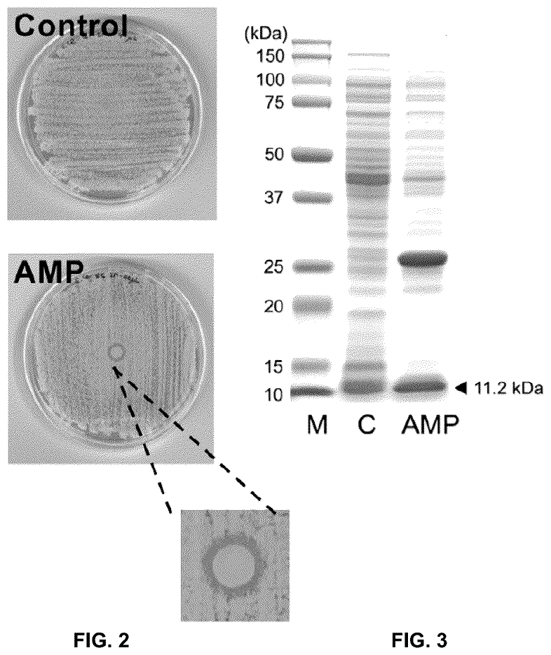

A potential novel antimicrobial peptide or AMP (PF09117, 96% eukaryal) is detected only in a small subset of soil- and plant-associated Actinobacteria outside of plant and fungal genomes (FIG. 1). Inhibition of Saccharomyces cerevisiae is demonstrated by an AMP candidate from Streptosporangium becharense DSM 46887 cloned into E. coli (see STAR Methods; FIG. 2). A potential dimeric form of the AMP is suggested by the presence of an about 25 kDa band in addition to the expected 11.2 kDa product on an SDS-PAGE gel (FIG. 3). AMP dimerism has been previously reported (1, 2) The sequence lengths of 59 candidate actinobacterial AMPs varied from 101 to 121 amino acids with a median length of 102 residues. An N-terminal signal peptide was detected in every instance. A survey of gene neighborhoods revealed no conserved colocalized functions. AMPs are a promising new class of therapeutic antibiotics displaying broad-spectrum antimicrobial efficacy against bacteria, fungi, and viruses (3-5).

A 102 amino acid AMP candidate from Streptosporangium becharense DSM 46887 (GenBank ID: MBB5817359.1) with a predicted molecular weight of 11.2 kDa, pI of 8.67 and charge of 3.5, was cloned in E. coli for functional validation. The sequence was codon optimized and sent to Twist Biosciences (San Francisco, CA) for synthesis and cloned into a pET-21 (+) expression vector. The plasmid DNA (pET-21 (+)_AMP_S.be) was then transferred into E. coli BL21 (DE3) strain with 100 mg/mL carbenicillin (MilliporeSigma, Burlington, MA) selection. To overexpress the short peptide, 100 mL of E. coli BL21 (DE3) harboring the recombinant plasmid was cultured in Luria-Bertani broth containing 100 mg/mL of carbenicillin at 37° C. until the mid-exponential phase. The overexpression was induced by addition of 0.1 mM isopropyl-b-D-thiogalactopyranoside (MilliporeSigma, Burlington, MA) at 25° C. and 120 rpm for 16 h. The cells were harvested by centrifugation at 6,000 rpm and 4° C. for 10 min and protein was extracted with BugBuster_Protein Extraction Reagent (MilliporeSigma, Burlington, MA) following the protocol from the kit. The protein was concentrated using two different Amicon Ultra centrifugal filters (MWCO 3 kDa and 30 kDa, MilliporeSigma, Burlington, MA).

Yeast Saccharomyces cerevisiae was used for the antimicrobial activity assessment with proteins extracted from the recombinant E. coli. S. cerevisiae was streaked and cultivated on the YPD agar plate at 30° C. one day prior to the assay. One or a few yeast colonies were resuspended in 2 mL of 0.85% sterile saline with a sterile inoculating loop. A sterile swab was dipped into the inoculum tube and was rotated to remove the excess fluid. The swab was then streaked on the YPD plate while rotating to distribute the inoculum evenly. 6-mm sterilized filter paper disk soaked with 20-40 mL of the protein extract was placed on the YPD agar plate. Fluconazole (25 mg/disk) was used for positive control. The protein extract obtained from E. coli BL21 (DE3) harboring empty pET-21 (+) plasmid was used as negative control. After incubation at 30° C. for 24 h, the antimicrobial activity of the short peptide was determined by appearance of the zone of inhibition. To confirm the overexpression of the short peptide, the protein extract was analyzed using SDS-PAGE gel (12% Mini-PROTEAN_TGXTM Precast Gel, Bio-Rad Laboratories, Hercules, CA). The same amount (20 mg) of each protein extract from E. coli BL21 (DE3) harboring the recombinant plasmid or empty plasmid was loaded in a single well and the gel was run at 180 V for 40 min. The gel was stained using Coomassie Brilliant Blue R-250 Staining Solution (Bio-Rad Laboratories, Hercules, CA).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A composition comprising an isolated or purified antimicrobial peptide having an amino acid sequence having at least 70% amino acid identity with one of SEQ ID NO: 1-95; wherein the antimicrobial peptide is capable of killing or inhibiting growth of a fungal or bacterial species.

2. The composition of claim 1, wherein the amino acid sequence has at least 80% amino acid identity with one of SEQ ID NO:1-95.

3. The composition of claim 2, wherein the amino acid sequence has at least 90% amino acid identity with one of SEQ ID NO:1-95.

4. The composition of claim 3, wherein the amino acid sequence has at least 95% amino acid identity with one of SEQ ID NO:1-95.

5. The composition of claim 4, wherein the amino acid sequence has at least 99% amino acid identity with one of SEQ ID NO:1-95.

6. The composition of claim 1, wherein the amino acid sequence comprises SEQ ID NO: 96, SEQ ID NO:98, or SEQ ID NO:100.

7. The composition of claim 6, wherein the amino acid sequence has at least 70% amino acid identity with one of SEQ ID NO:1-6, 8-11, 20, 25, 27, 30, 33-35, 45-48, 50, 52-56, 58-60, 62-71, 73-77, 81. 83, 84, 86, 89, 91, or 93.

8. The composition of claim 7, wherein the amino acid sequence comprises SEQ ID NO: 96, and has at least 70% amino acid identity with one of SEQ ID NO:1-6, 8, 10, 11, 20, 27, 30, 33-35, 45-48, 52-56, 58-60, 62-66, 68, 70, 71, 73-77, 81. 83, 86, 91, or 93.

9. The composition of claim 8, wherein the amino acid sequence comprises SEQ ID NO: 97.

10. The composition of claim 7, wherein the amino acid sequence comprises SEQ ID NO: 98, and has at least 70% amino acid identity with one of SEQ ID NO: 1, 2, 4-6, 9, 25, 27, 30, 34, 54, 55, 59, 60, 62-65, 67, 69, 71, 73, 76, 77, 81, 84, 89, or 91.

11. The composition of claim 10, wherein the amino acid sequence comprises SEQ ID NO: 99.

12. The composition of claim 7, wherein the amino acid sequence comprises SEQ ID NO: 100, and has at least 70% amino acid identity with one of SEQ ID NO:1-6, 8-11, 20, 25, 27, 30, 33-35, 45, 47, 48, 50, 53-56, 58-60, 62-67, 69-71, 73-77, 81, 83, 84, 86, 91, or 93.

13. The composition of claim 12, wherein the amino acid sequence comprises SEQ ID NO: 101.

14. The composition of claim 7, wherein the amino acid sequence has at least 70% amino acid identity with SEQ ID NO:6.

15. The composition of claim 1, wherein the antimicrobial peptide is about 100 amino acid residues to about 220 amino acid residues long.

16. The composition of claim 15, wherein the antimicrobial peptide is about 100 amino acid residues to about 130 amino acid residues long.

17. The composition of claim 16, wherein the antimicrobial peptide is about 100 amino acid residues to about 120 amino acid residues long.

18. The composition of claim 1, wherein the antimicrobial peptide is capable of killing or inhibiting growth of a Gram-positive bacterial species.

19. A method of inhibiting growth of a fungal or bacterial cell, comprising contacting an isolated or purified antimicrobial peptide having an amino acid sequence having at least 70% amino acid identity with one of SEQ ID NO:1-95, or a composition comprising the isolated or purified antimicrobial peptide, with a fungal or bacterial cell.


SEQUENCE LISTING (107 sequences)

SEQ ID NO: 1 - protein, 102 aa, Nonomuraea muscovyensis
VKRIALVPVISAAMVAIMGSAAYASQYVAYEGPGFTGRSVVIDRCGPSNIPFHGSYKWYGDGQSGRMYNQRDTQGVMHFELPSNRNAEQRTPVGWQSIFIVC

SEQ ID NO: 2 - protein, 102 aa, Streptosporangium saharense
VKRVLFVSALTAGIVAITTGAAYASQYIAYEGRGFTGRSVVIDACGPSNIPYHGSYKWYGDGQSGRMYNQPNAQGLMHFKLASDRNAEQRTPVGWQSIFIEC

SEQ ID NO: 3 - protein, 102 aa, Microbispora rosea
MKRFVLVPALTAATIAISAGAAYASQYVAYEGPGFSGRSVVIDRCGVSNIPFHGSYRWYGDGQSGRMYNQPDAQGVAHFTLAADRNAEQRTPVGWKSIFIVC

SEQ ID NO: 4 - protein, 102 aa, Sphaerisporangium cinnabarinum
VKRLVLVPVISAAMLTVMAGAAYASQYVAYEGPAFTGRSVVIDRCGPSNIPFHGSYKWYGDGQSGRMYNQPDTQGVMHFELASNRYAEQRTPVGWKSIFIVC

SEQ ID NO: 5 - protein, 102 aa, Nonomuraea roseoviolacea
VKRIALVPVIAAAMVAVMGSAAYASQYVAYEGPGFTGRQQFIDACGPSNIHYHGSYKWYGDGQSGRMYNQPNAQGVMHFELAADRNAEQRTPVGWQSIFIVC

SEQ ID NO: 6 - protein, 102 aa, Streptosporangium becharense
MKRIFLVPALTAGIFALTTGAAYASQYIAYEGPGFTGRSVLIDACGPSNIPYHGSYKWYGDGQSGRMYNQPNAHGVMHFRLPSNNNAEQRTPVGWQSIFIEC

SEQ ID NO: 7 - protein, 106 aa, Actinomadura catellatispora
MNRKRLALVPALTAGIIACTAAAAQASTIVVYEGSHFSGESMTIDACGPSDIPERFHGSYKWYATGQSASMYNLPGGKGRVHHRLSAHWDNEQKTPFGWKSMIIHC

SEQ ID NO: 8 - protein, 102 aa, Microbispora bryophytorum
MKRFVLVPALTAATIAISAGAAYASQYVAYEGPGFSGRSVVIDRCGMSNIPFHGSYRWYGDGQSGRMYNQPDAQGVAHFTLAADRNAEQRTPVGWKSIFIVC

SEQ ID NO: 9 - protein, 102 aa, Nonomuraea jiangxiensis
MKRILLVPAIAAGIIAITAGAAQASQYVAYEGRGFTGRSVVLDACGVSNIPFHGSYKWYGDGQSGRMYNQPNAQGVAHFTLPSNRNAEQRTPVGWKSIFIVC

SEQ ID NO: 10 - protein, 102 aa, Nonomuraea gerenzanensis
VKRFVTTAAFTAAIVVGAAGAAYASQYVAYEGPGFTGRSVVIDRCGLSNIPFNGSYKWYGDGQSGRMYNQPNAEGVAHFTLSSNKNAEQRTPVGWKSIFIVC

SEQ ID NO: 11 - protein, 102 aa, Microbispora rosea; identical to SEQ ID NO: 3

SEQ ID NO: 12 - protein, 107 aa, Streptomyces piniterrae
MRNQHTVRTTVTAAMTAAVLVMSTGAAFASSFRGWSGAHYTGTSSEVTRCGCSNLSLNHRGSYRFDHTGQDASMFNTRNCQGSPHYTFRGDASSPAPVGWRSIYIHC

SEQ ID NO: 13 - protein, 107 aa, Streptomyces piniterrae
MRNQHTVRTTVTAAMTAAVLAVSTGAAFASSFRGWSGAHYTGTSSEVTRCGCSNLSLGHRGSYRFDHTGQDASMFNTRNCQGSPHYTFRGDASSPAPVGWRSIYIHC

SEQ ID NO: 14 - protein, 107 aa, Streptacidiphilus albus
MRMFGHRRLLAVGAIAAAVVMGSQAAAFASDFIAYPGPGYSGQAIDLSACGYNRIPHGYNGSYVFQYTGQTADAYNNPDASGVAAFRFTGDAQQDTPYGWQSVWIQC

SEQ ID NO: 15 - protein, 121 aa, Streptomyces ansochromogenes
MKEIVIEMKFTLRTAVALAATAAVSGLAAVPATATEHQARDMTSSFVAYEGTSFSGRSADINGCGYHSIPFHGSYKWIPRGQDGRMHNAADGRDPVHTVLRSNRTAEQSTPFGWQSLYIVC

SEQ ID NO: 16 - protein, 118 aa, Streptomyces sp.
MRKFMFSRRAAAVLVATAAIGGAVAAAPATAAESRPAGMTSSFVAYPGTSFTGQPVDINGCGLHDIPAHGSYKWIARGQSGRMYDQPGAGGPVHTVLASDENAEQSTPFGWQSIFIVC

SEQ ID NO: 17 - protein, 108 aa, Streptomyces sp.
MRNRHTLRTTVISAVTSAVLALGSGAALASDWYGYPLSNQKGQPQHIRACGCSNLAEGKRASYYFDHRGQDATMYNDRDCPRGHGHYKFRGDSERNASFGWRSIEIHC

SEQ ID NO: 18 - protein, 117 aa, Streptomyces sp.
MRTFTFGRRAAAALVATVAIGGLAAVPSTAAESGAAGMTSSFVAYEGTSFSGRSADINGCGFHQIPFHGSYKWVPRGQDGRMHNAADGRDPVHTVLRSNRDAEQSTPFGWQSLYIVC

SEQ ID NO: 19 - protein, 108 aa, Streptomyces sp.
MRNRHTLRLAVTSAVTSAALALTSGAALASDWYGYPRSNQQGQPQHILACGCSNLDEGKRGSYYFDHRGQDATMFNDRDCPRGHGHYTFRGDSERNASFGWKSIHIHC

SEQ ID NO: 20 - protein, 102 aa, Nonomuraea sp.
VKKFVLTSVFTAAIVAGTAGAAYASQYVAYEGPGFTGRSVVIDRCGVSNIPFNGSYKWYGDGQSGRMYNQPNAEGVAHFTLASNRNAEQRTPVGWKSIFIVC

SEQ ID NO: 21 - protein, 107 aa, Streptacidiphilus albus; identical to SEQ ID NO: 14

SEQ ID NO: 22 - protein, 101 aa, Herbidospora sakaeratensis
VKRILLTSAIAAVIAGTAGMAHASVYVAYEGPGFSGRSVALSACGINNIPFGGSYKWYGTGQSGRMYNQPNAQGVAHFTLDSNRNAEQITPVGWKSVFIVC

SEQ ID NO: 23 - protein, 102 aa, Herbidospora yilanensis
VKRIILTSALAAAVVAGTAGMAHASVYVAYEGPGFSGRSVALSACGTNNIPFGGSYKWYGTGQSGRMYNQPNAEGTAHFTLDSNRNAEQITPVGWKSIFIVC

SEQ ID NO: 24 - protein, 101 aa, Herbidospora daliensis
VKRIILTSAIAAVIAGTAGMAHASVYVAYEGPGFSGRSVALSACGINNIPFGGSYKWYGTGQSGRMYNQPNAEGVAHFTLDSNRNAEQISPVGWKSVFIVC

SEQ ID NO: 25 - protein, 102 aa, Herbidospora cretacea
MKKIILTSALAAAFTLGTAGAALASQYVAYEFPGFAGRSVVISACGMSNIPFHGSYKWYGDGQSGRMYNQPNAQGVAHFTLDSARNAEQRTPVGWQSIFIVC

SEQ ID NO: 26 - protein, 110 aa, Streptomyces scopuliridis
MRNRRNRRTLRSTVTATAVAGVLALGAGTAVASGFLSWEGPQNTGRAKELTRCGCSNIDPNFRAAYRFTYTGQTAAMYNQPDCAGTSHYTFRGNSESVQPFGWRSIFIHC

SEQ ID NO: 27 - protein, 102 aa, Streptosporangium amethystogenes
VKRIALVPVISAAMVAVMGSAAYASQYVAYEGPGFTGRSVVIDRCGPSNIPFHGSYKWYGDGQSGRMYNQPNTQGVTHFELPSNRNAEQRTPVGWKSIFIVC

SEQ ID NO: 28 - protein, 101 aa, Herbidospora cretacea
VKRILLTSALAAVIAGTAGMAHASVYVAYEGPGFSGRSVALSACGINNIPFGGSYKWYGTGQSGRMYNLPNAQGVAHFTLDSNRNAEQITPVGWKSVFIVC

SEQ ID NO: 29 - protein, 117 aa, Streptomyces lilacinus
MLNSKFSRRAAAALAATAVIGGLTAVPATATESRAYGMTSSFVAYEGTSYSGRSADINGCGYHSIPFHGSYKWIPRGQDGRMHNAADGRDPVHTVLRSNRDAEQSTPFGWQSIYIVC

SEQ ID NO: 30 - protein, 102 aa, Nonomuraea indica
VKRLALVPVISAAMVAIMGTAAYASQYVAYEGPGFTGRSVVIDRCGPSNIPFHGSYKWYGDGQSGRMYNQRDTQGVMHFELPSDRNAEQRTPVGWQSIFIVC

SEQ ID NO: 31 - protein, 108 aa, Streptomyces lushanensis
MRNRHTLRLAVTSAVTSAVLALTSGAALASDWYGYPQSNQKGQPQHILACGCSNLDEGKRGSYYFDHRGQDATMFNDRDCPRGHGHYTFRGDSERNASFGWKSIHIHC

SEQ ID NO: 32 - protein, 107 aa, Streptomyces barkulensis
MRNQHTVRTTVIAAMTAAVLAVSTGAAFASSFRGWSGAHYTGTSSEVTGCGCSNLSIGHRGSYRFDHTGQDASMFNTRNCQGSPHYTFRGDASSPAPVGWRSIYIHC

SEQ ID NO: 33 - protein, 102 aa, Microbispora sp.
MKRFVLVPALTAATIAISAGAAYASQYVAYEGPGFSGRSVVIDRCGVSNIPFHGSYRWYGDGQSGRMYNQPDAQGVAHFTLAADRNAEQRTPVGWQSIFIVC

SEQ ID NO: 34 - protein, 102 aa, Nonomuraea sp.
MKKVLLVPAIAAGIVLASAGAAYASQYVAYEGPGFTGRSVVLDACGVSNIPYHGSYKWYGDGQSGRMYNQPNATGVTHFELASNRNAEQRTPVGWKSIFIVC

SEQ ID NO: 35 - protein, 102 aa, Actinomadura parvus; identical to SEQ ID NO: 20

SEQ ID NO: 36 - protein, 107 aa, Actinobacteria sp.
MKARHAVRSAVTASVAAAVVVLGTGSAYASSFLSWEGPQNTGRAKVVHACGCNNIDDNFKAAYRFTYTGQTAAMYNEPDCKGLVHHTFRGSQESKDPFGWRSIFFHC

SEQ ID NO: 37 - protein, 106 aa, Actinobacteria sp.
MNRKRLALLPALTAGIIACTAAAAQASTIVVYEGSHFTGASMTIDACGPSDIPERFHGSYKWYATGQSASMYNLPGGKGRVHHKLSAHWDNEQKTPFGWKSMIIHC

SEQ ID NO: 38 - protein, 106 aa, Actinobacteria sp.
VRKKTMALVPALTAGIIAVSAAAAQASTIVVYEGDHYSGRSMTINKCGMSNIPEAFHGSYKWYATGQSARMYNQPNAEGVMHYELSARHNNEQATPFGWKSMFIVC

SEQ ID NO: 39 - protein, 101 aa, Nonomuraea sp.
MSLVLVPALAAGLITFAPTTAQASTFVVYEGSHYTGRSAALSACGMSNIPYNGSYKWYATGQSARMYNQPNGVGVGHYTFSASRSGERPEGFGWKSIIIHC

SEQ ID NO: 40 - protein, 107 aa, Streptomyces sp.
MRIRRTLRTAVTSAVMTGVFVLGTGVASASSFLSWEGPQNTGRAKEVFACGCNNIDPNFRAAYRFKHTGQDATMYNQDNCQGQSHFTFHGSQESRDPFGWRSVFFHC

SEQ ID NO: 41 - protein, 107 aa, Streptomyces aurantiacus
MRTRHALRTAVSATMLAGALLLGTSPAFASSFLSWAGPQNTGSAKEVFACGCNNIDPGHKAAYRFTYTGQTAAMYNESNCQGTVHHTFRGSQESRDGFGWKSVFFHC

SEQ ID NO: 42 - protein, 107 aa, Streptomyces niveus
MRKRHALRTAVSATMLAGALVLGSGTAIASSFLSWSGPQNTGSAREVFACGCNEIDPGHKAAYRFTYTGQTAAMYNEEGCRGKVHTTFRGSQESRGAFGWKSVFFHC

SEQ ID NO: 43 - protein, 101 aa, Herbidospora galbida
VKRIILTSALAAVIAGTAGIAHASVYVAYEGPGFSGRSVALTACGTSNIPFGGSYKWYGTGQSGRMYNQPNAEGTAHFTLDSNRNAEQVTPVGWKSIFIVC

SEQ ID NO: 44 - protein, 101 aa, Herbidospora sp.
VKRILLTSALAAVIAGTAGMAHASVYVAYEGPGFSGRSVALSACGINNIPFGGSYKWYGTGQSGRMYNQPNAQGVAHFTLDSNRNAEQITPVGWKSVFIVC

SEQ ID NO: 45 - protein, 102 aa, Microbispora catharanthi
MRRFVLVPALTAATIAISAGAAYASQYVAYEGPGFSGRSVVIDRCGVSNIPFHGSYRWYGDGQSGRMYNQPDAQGVAHFTLAADRNAEQRTPVGWKSIFIVC

SEQ ID NO: 46 - protein, 102 aa, Microbispora sp.
MRRIVLIPALTAATIAISAGAAYASQYVAYEGPGFSGRSVVIDRCGVSNIPFHGSYRWYGDGQSGRMYNQPDAQGVAHFTLAADRNAEQRTPIGWKSIFIVC

SEQ ID NO: 47 - protein, 102 aa, Microbispora sp.
MRRIALVPALTAATIVATAGAAYASQYVAYEGPGFSGRSVVIDACGTSNIPYHGSYRWYGDGQSGRMYNQPNAQGVAHFTLPSDRNAEQRTPVGWQSIFIVC

SEQ ID NO: 48 - protein, 102 aa, Nonomuraea sp.
VKKFVLTSVFTAAIVAGTAGAAYASQYVAYEGPGFTGRSVVIDRCGVSNIPFNGSYKWYGDGQSGRMYNQPNAQGVAHFTLASNRNAEQRTPVGWKSIFIVC

SEQ ID NO: 49 - protein, 112 aa, Nonomuraea sp.
VKSIKRIALVSAISASLVTIMGGAANASISAAPGSMYVAYEGPGFTGRTQVISQCGTTNLRYRGSYKWYGSGQAGRMHNQINARGPVHFKLPTKGNAQQNKAVGWKSIFKVC

SEQ ID NO: 50 - protein, 102 aa, Nonomuraea sp.
MKRVTLIPALTAGIIALTAGAAHASQYIAYEGPGFTGRSVLIDACGMSNIPFNGSYKWYGDGQSGRMYNQPNAEGLMHFKLPADNSAEQRTPVGWKSIFIEC

SEQ ID NO: 51 - protein, 107 aa, Streptomyces ficellus
MRIRRTLRTAITSTVMTGVFVLGTGVASASSFLSWEGPQNTGRAKEVFACGCNNIDPNFRAAYRFKHTGQDATMYNQDNCQGQSHFTFHGSQESRDPFGWRSVFFHC

SEQ ID NO: 52 - protein, 102 aa, Microbispora sp.
MKRYVLVPALTAATIAISAGAAYASQYVAYEGPGFSGRSVVIDRCGVSNIPFHGSYRWYGDGQSGRMYNQPDAQGVAHFTLAADRNAEQRTPVGWKSIFIVC

SEQ ID NO: 53 - protein, 102 aa, Microbispora sp.; identical to SEQ ID NO: 3

SEQ ID NO: 54 - protein, 102 aa, Nonomuraea sp.
VKRIALVPVLAAAMVAVMGSAAYASQYIAYEGPGFTGRQQFIDACGPSNIHYHGSYKWYGDGQSGRMYNQPNAQGVMHFELASNRNAEQRTPVGWQSIFIVC

SEQ ID NO: 55 - protein, 102 aa, Nonomuraea rhodomycinica
VKRIALVPVLAAAMVAVMGSAAYASQYIAYEGPGFTGRQQFIDACGPSNIHYHGSYKWYGDGQSGRMYNQPNAQGVMHFELAADRNAEQRTPVGWQSIFIVC

SEQ ID NO: 56 - protein, 218 aa, Nonomuraea sp.
VLRWQARAVGARGDRQDREEWLEIDRSQRKHAHRGSRLEAFYASESTVLRVLQAAGMTVVAPIVSVVPTGNCPQRDQSDTLATQLRPMARKLLGRLHAVALLSPCVSIEWGLGRIIVKKFILTSAFTAAIVVGTAGAAYASQYVAYEGPGFTGRSVVIDRCGVSNIPFNGSYKWYGDGQSGRMYNQPNAEGVAHFTLASNRNAEQRTPVGWKSIFIVC

SEQ ID NO: 57 - protein, 108 aa, Actinobacteria sp.
MRNRHTLRLAVTSAVTSAVLALTSGAALASDWYGYPLSNQKGQPQHIVACGCSNLNEGKRGSYYFDHRGQDATMYNDRDCPRGHGHYTFRGDSERNASFGWKSIEIHC

SEQ ID NO: 58 - protein, 102 aa, Microbispora sp.
MKRFFLVPALTAATIAISAGAAYASQYVAYEGPGFSGRSVVIDRCGMSNIPFHGSYRWYGDGQSGRMYNQPDAQGVAHFTLAADRNAEQRTPVGWKSIFIVC

SEQ ID NO: 59 - protein, 102 aa, Actinomadura sp.
LKKILLVPAITAGFVLASAGAAYASQYVAYEGPGFTGRSVVLDACGVSNIPYHGSYKWYGDGQSGRMYNQPNASGVMHFELASNRNAEQRTPVGWKSIFIVC

SEQ ID NO: 60 - protein, 102 aa, Actinobacteria sp.; identical to SEQ ID NO: 59

SEQ ID NO: 61 - protein, 104 aa, Actinobacteria sp.
MRKRSLILVPALAAGLITCSATTAQASTFVVYEGSHYTGRSVGLNACGMSNIPYNGSYKWYATGQSARMYNQPNGQGVGHYTFSGSRSGERPEGFGWKSIIIHC

SEQ ID NO: 62 - protein, 102 aa, Actinobacteria sp.
VKRIALVPVISAALVAVMGSAAYASQYVAYEGPGFTGRSAIIDRCGPSNIPFHGSYKWYGDGQSGRMYNQPDAQGVMHFELASDRNAEQRTPVGWKSIFIVC

SEQ ID NO: 63 - protein, 102 aa, Actinobacteria sp.
VKRIALVPLISAAMVVVMGGAAYASQYVAYEGPGFTGRSVVIDRCGPSNIPFHGSYKWYGDGQSGRMYNQPDAQGVTHFELASNRNAEQRTPVGWKSIFIVC

SEQ ID NO: 64 - protein, 103 aa, Actinobacteria sp.
MLKKILLVPAITAGLVLASAGAAYASQYVAYEGRGFTGRSVVLDACGVSNIPYHGSYKWYGDGQSGRMYNQPNATGVMHFELASNRNAEQRTPVGWKSIFIVC

SEQ ID NO: 65 - protein, 102 aa, Actinobacteria sp.; identical to SEQ ID NO: 34

SEQ ID NO: 66 - protein, 102 aa, Actinobacteria sp.
VKRIALVPVISAALVVVMGSAAYASQYVAYEGPGFTGRSVVIDRCGPSNIPFHGSYRWYGDGQSGRMYNQPDTQGVMHFELASNRNAEQRTPVGWKSIFIVC

SEQ ID NO: 67 - protein, 102 aa, Actinobacteria sp.
MKRIAQVITAAAMTVVATAGTAVASQYVAYEGEGFSGRSVVITACGTSNIPYHGSYKWYGDGQSGRMYNQSNAQGVAHFTLASNRYAEQRTPVGWKSIFIVC

SEQ ID NO: 68 - protein, 102 aa, Actinobacteria sp.
VKRIALVPVLAAAMVAIMGSAAYASQYIAYEGPGFTGRTQFIDACGVSNIVYHGSYKWFGDGQSGRMYNQPNARGPMHFELASNRNAEQHTSVGWQSIFIVC

SEQ ID NO: 69 - protein, 102 aa, Actinobacteria sp.
VKRIALVPVISAALVVVMGSAAYASQYVAYEGPGFTGRSVVIDRCGPSNIPFHGSYKWYGDGQSGRMYNQPDTQGVMHFELASNRNAEQRTPVGWKSIFIVC

SEQ ID NO: 70 - protein, 102 aa, Actinobacteria sp.
MKRILLVPALTAGIIALTAGAAYASQYIAYEGPGFTGRSVLIDACGPSNIPFHGSYKWFGDGQAGRMYNQPNTQGPMHFRLPADDGAEQRTPVGWQSIFIEC

SEQ ID NO: 71 - protein, 102 aa, Actinobacteria sp.; identical to SEQ ID NO: 59

SEQ ID NO: 72 - protein, 112 aa, Actinobacteria sp.
VKSIKKIALVSAISASLITIMGGAANASTSAAPDSMYVAYEGPGFTGRTQVITECGLTNLRYRGSYKWYGAGQSGRMHNQINARGSVHFTLPTNSNAQQKTGIGWKSIFKVC

SEQ ID NO: 73 - protein, 102 aa, Actinobacteria sp.
VKRILLVPALTAGIIALTASAAYASQYVAYEGRGFTGRSVVLDACGVSNIPFHGSYKWYGDGQSGRMYNQPNAQGVMHFELPSNRNAEQRTPVGWQSIFIVC

SEQ ID NO: 74 - protein, 102 aa, Actinobacteria sp.; identical to SEQ ID NO: 20

SEQ ID NO: 75 - protein, 102 aa, Actinobacteria sp.; identical to SEQ ID NO: 3

SEQ ID NO: 76 - protein, 102 aa, Actinobacteria sp.
VKRTLIVSALTAGIVAITAGAAYASQYVAYEGPGFTGRSVVIDACGPSNIPYHGSYKWYGDGQSGRMYNQPDAQGLMHFRLPSDDNAEQRTPVGWQSIFIEC

SEQ ID NO: 77 - protein, 102 aa, Actinobacteria sp.; identical to SEQ ID NO: 59

SEQ ID NO: 78 - protein, 130 aa, Actinobacteria sp.
MEHSLRRRPRSRTRLRATCLALFGAAAIAAPVAIASPANAAPAPARLVLSATSTFVAYSGGSFSGDSQGIQGCGSHTIRYHGSYKWYANGQSGHLFNNSNTSGVPHTRLASDDDAESRSGFAWRSILIVC

SEQ ID NO: 79 - protein, 109 aa, Actinobacteria sp.
VKRIILVSAVATALVTAMSGAAYASTSAGYSSTYVAFEGPGFTGRTQVITRCGTTNIRYRGSYKWFGGGQDGRMHNDINARGPVHFQLASNRNARQETGIGWKSIFKVC

SEQ ID NO: 80 - protein, 100 aa, Actinobacteria sp.
VRPVVAAGATAALFVVGTGMAFASRFESYEKANYSGAASTVAACGCNSIEYGGSYIFEYSGQTARVYKQKGCKGRAAGDLTHNSRNPDTVGGWKSIFILC

SEQ ID NO: 81 - protein, 102 aa, Actinobacteria sp.
VKRLALVPVISAAMIALMGTAAYASQYVAYEGPAFTGRSVVIDRCGPSDIPFHGSYKWYGDGQSGRMYNQPGTHGVMHFELASNRYAEQRTPVGWQSIFIVC

SEQ ID NO: 82 - protein, 129 aa, Actinobacteria sp.
MENSHRRRPRSGTRLRATCLALLGAASIATPLIASPANAVSAPARLALSATSTFVAYSGSSFSGDSEGIQGCGLHTISYHGSYKWYANGQSGHLFNNSTGSGVPHTRLASDDNAESGSGFAWGSILIVC

SEQ ID NO: 83 - protein, 104 aa, Actinobacteria sp.
GAVKRFLLVPAMTAAIIAITAGAAYASQYIAYEGRGFTGRSVVIDACGVSNIPFNGSYKWYGDGQSGRMYNQRDAKGVMHFELASNRNAEQRTPVGWKSIFIVC

SEQ ID NO: 84 - protein, 102 aa, Actinobacteria sp.
MKRVAFVSAATAALVAIMGSAAFASQYVAYEGPGFSGRSVTIDKCGTSNIPFHGSYKWYGDGQSGRMYNQPNAEGVMHFELASNRYAEQRTPVGWQSIFIVC

SEQ ID NO: 85 - protein, 107 aa, Actinobacteria sp.
MRHQHTVRTTVTAAVTAAVLVMSTGAAFASSFRGWSGAHYTGTSSEVTRCGCSNLSLNHRGSYRFDHTGQDASMFNTRNCQGSPHYTFRGDSSSPAPVGWRSIYIHC

SEQ ID NO: 86 - protein, 102 aa, Actinobacteria sp.
MKRFLLVSALTAATIAISAGAAYASQYVAYEGPAFTGRSVVIDKCGVSNIPFHGSYRWYGDGQSGRMYNQPNAEGVAHFTLPSDRNAEQRTPVGWKSIFIVC

SEQ ID NO: 87 - protein, 117 aa, Actinobacteria sp.
MPKFAFGRRAAAALVATVAIGGLSAVPSTAAEAGADGMTSSFVAYEGTSFSGRSADINGCGYHQIPFHGSYKWVPRGQDGRMHNAADGRDPVHTVLRSNRGAEQSTPFGWQSIYIVC

SEQ ID NO: 88 - protein, 107 aa, Actinobacteria sp.
MRKRRSLRTAVGSLMLSGALVLGTGTSFASSFLSWEGPQNTGRAKEVHACGCNNIDPNYRAAYRFTYTGQTAAMYNEPDCRGLVHHTFRGSQESKDAFGWRSVFFHC

SEQ ID NO: 89 - protein, 102 aa, Actinobacteria sp.
MKRFPVALAAAAMSVVAAAGSAAASQLVVYEGEGYSGRSHVITACGASNIPYHGSYKWYGDGQSGRMYNQPNAQGVAHFTLPSDRNAGQRTPIGWRSIFIVC

SEQ ID NO: 90 - protein, 106 aa, Actinobacteria sp.
MRKKTLALAPAVTAGIIALSAAAAQASTIVVYEGDHFSGHSITLSKCGMSNIPARYHGSYKWYATGQSARMYNQPNAEGVVHFVLRSDRNAEQATPFGWRSMFIEC

SEQ ID NO: 91 - protein, 102 aa, Actinobacteria sp.
MKRILLVPALTAGIIALTASAAYASQYIAYEGRGFTGRSVVLDACGVSNIPFHGSYKWYGDGQSGRMYNQPNAQGVMHFELPSNRNAEQRTPVGWQSIFIVC

SEQ ID NO: 92 - protein, 102 aa, Actinobacteria sp.
MKRVSLVLAAAAMSVVAAAGSAAASQLVVYEGEGFSGRSAVISACGTSNLPYHGSYKWYSDGQSGRMYNQRDAQGVANFTLAPDRNAEQRTSVGWQSIFIVC

SEQ ID NO: 93 - protein, 102 aa, Actinobacteria sp.
MKKLAVVPVISAAMLAVMASAAYASQYVAYEGQAFTGRSVVIDRCGVSNIPFGGSYKWYGDGQSGRMYNQPNAEGVMHFELRSNRNAEQRTPVGWKSIFIVC

SEQ ID NO: 94 - protein, 123 aa, Actinobacteria sp.
MIIPRTAQQATSIRRVAAALAAAAAIGGLTAVPATATESRAYGMTSSFVAYEGTSFSGRSADINGCGQHAIPFHGSYKWIPRGQDGRMHNAADGRDPVHTVLRSNRDAEMSTPFGWQSIYIVC

SEQ ID NO: 95 - protein, 107 aa, Actinobacteria sp.; identical to SEQ ID NO: 40

SEQ ID NO: 96 - protein, 7 aa, Nonomuraea muscovyensis
AAYASQY

SEQ ID NO: 97 - protein, 18 aa, Nonomuraea muscovyensis
AAYASQYXAYEGPXFTGR

SEQ ID NO: 98 - protein, 18 aa, Nonomuraea muscovyensis
HGSYKWYGDGQSGRMYNQ

SEQ ID NO: 99 - protein, 29 aa, Nonomuraea muscovyensis
IDXCGXSNIXXHGSYKWYGDGQSGRMYNQ

SEQ ID NO: 100 - protein, 9 aa, Nonomuraea muscovyensis
AEQRTPVGW

SEQ ID NO: 101 - protein, 16 aa, Nonomuraea muscovyensis
AEQRTPVGWXSIFIXC

SEQ ID NO: 102 - protein, 5 aa, Streptomyces pini
AAXAS

SEQ ID NO: 103 - protein, 6 aa, Streptomyces pini
AXXAAV

SEQ ID NO: 104 - protein, 7 aa, Streptomyces pini
AAXASXF

SEQ ID NO: 105 - protein, 5 aa, Streptomyces pini
GSYXF

SEQ ID NO: 106 - protein, 5 aa, Streptomyces pini
TGQXA

SEQ ID NO: 107 - protein, 16 aa, Streptomyces pini
GDAXXXXPXGWXSXXI